(19) United States
(12) Patent Application Publication
(10) Pub. No.: US 2024/0212200 A1
(43) Pub. Date: Jun. 27, 2024

(54) SENSOR ALIGNMENT FOR A NON-CONTACT PATIENT MONITORING SYSTEM

(71) Applicant: Covidien LP, Mansfield, MA (US)

(72) Inventors: Dean MONTGOMERY, Edinburgh (GB); Paul S. ADDISON, Edinburgh (GB)

(21) Appl. No.: 18/332,389

(22) Filed: Jun. 9, 2023

Related U.S. Application Data
(60) Provisional application No. 63/367,180, filed on Jun. 28, 2022.

(51) Int. Cl. G06T 7/73; G06T 7/00; G06T 7/50; G06T 7/62; G06V 10/25; G06V 10/776

(52) U.S. Cl. CPC G06T 7/73; G06T 7/0012; G06T 7/50; G06T 7/62; G06V 10/25; G06V 10/776; G06T 2207/30061

(57) ABSTRACT

The disclosed technology qualifies sensor alignment relative to a patient environment by determining a respiratory region of the patient, detecting, by the sensor, sensor data including a plurality of distances between a position of the sensor and the respiratory region, generating, based at least in part on the detected sensor data, an aggregate alignment metric, determining that the aggregate alignment metric satisfies a misalignment condition, classifying a characteristic of misalignment of the sensor relative to the determined respiratory region at least partially based on the satisfaction of the misalignment condition, and generating an instruction to provide a misalignment notification based at least partially on the classified characteristic of misalignment.

### CROSS REFERENCE TO RELATED APPLICATIONS

This application claims priority to U.S. Provisional Patent Application No. 63/367,180, filed Jun. 28, 2022, entitled SENSOR ALIGNMENT FOR A NON-CONTACT PATIENT MONITORING SYSTEM, the entirety of which is hereby incorporated by reference.

### TECHNICAL FIELD

The present disclosure relates to informative displays for non-contact patient monitoring, and more specifically, to informative displays for informing a user as to an alignment of a sensor relative to a patient environment. The information may be calculated from depth measurements taken and/or images captured by a non-contact patient monitoring system, including a depth-sensing camera, and an instruction for display of the alignment information representing the alignment of the sensor relative to the patient environment may be derived from the information. The information may include classifications representing a characteristic of misalignment of the sensor relative to the patient environment. The information may be derived relative to a region of interest (hereinafter, “ROI”) detected by the non-contact patient monitoring system, the ROI including a portion of the patient environment. The non-contact patient monitoring system may provide instructions to display the alignment information on a display device or on a sensor.

### BACKGROUND

Depth sensing technologies have been developed that, when integrated into non-contact patient monitoring systems, can be used to determine a number of physiological and contextual parameters, such as respiration rate, tidal volume, minute volume, etc. Such parameters can be displayed on a display so that a clinician is provided with a basic visualization of these parameters. For example, respiratory rate measurements may be presented.

However, the validity of physiological and contextual parameters is dependent upon the alignment of the sensor (e.g., a depth-sensing camera) relative to an ROI in the patient environment. If the sensor is misaligned relative to the ROI, it is likely that the physiological and contextual parameters derived from the sensor measurements will be incorrect and potentially unusable.

### SUMMARY

In some embodiments, the disclosed technology qualifies sensor alignment relative to a patient environment by determining a respiratory region of the patient, detecting, by the sensor, sensor data including a plurality of distances between a position of the sensor and the respiratory region, generating, based at least in part on the detected sensor data, an aggregate alignment metric, determining that the aggregate alignment metric satisfies a misalignment condition, classifying a characteristic of misalignment of the sensor relative to the determined respiratory region at least partially based on the satisfaction of the misalignment condition, and generating an instruction to provide a misalignment notification based at least partially on the classified characteristic of misalignment.

In some embodiments, the disclosed technology qualifies sensor alignment relative to a patient environment by determining a respiratory region of the patient, detecting, by the sensor, sensor data including one or more of a captured image of the respiratory region and a plurality of distances between a position of the sensor and the respiratory region, determining, by a predefined relationship between sensor data and sensed qualities of the respiratory region, a sensed quality of the determined respiratory region based on the detected sensor data, determining whether the sensed quality of the determined respiratory region satisfies a misalignment condition, classifying a characteristic of misalignment of the sensor relative to the determined respiratory region at least partially based on satisfaction of the misalignment condition, and generating an instruction to provide a misalignment notification based at least partially on the classified characteristic of misalignment.

In some embodiments, the disclosed technology provides a system for qualifying sensor alignment relative to a patient environment. The system includes one or more hardware processors, a sensor operable to detect sensor data, including a plurality of distances between a position of the sensor and a determined respiratory region of the patient, and an alignment manager executable by the one or more hardware processors. The alignment manager is configured to generate, based at least in part on the detected sensor data, an aggregate alignment metric, determine that the aggregate alignment metric satisfies a misalignment condition, classify a characteristic of misalignment of the sensor relative to the determined respiratory region at least partially based on the satisfaction of the misalignment condition, and generate an instruction to provide a misalignment notification based at least partially on the classified characteristic of misalignment.

In some embodiments, the disclosed technology provides a system for qualifying sensor alignment relative to a patient environment. The system includes one or more hardware processors, a sensor operable to detect sensor data, including one or more of a captured image of a determined respiratory region of the patient and a plurality of distances between a position of the sensor and the determined respiratory region. The system further includes an alignment manager executable by the one or more hardware processors. The alignment manager is configured to determine, by a predefined relationship between sensor data and sensed qualities of the respiratory region, a sensed quality of the determined respiratory region based on the detected sensor data, determine whether the sensed quality of the determined respiratory region satisfies a misalignment condition, classify a characteristic of misalignment of the sensor relative to the determined respiratory region at least partially based on satisfaction of the misalignment condition, and generate an instruction to provide a misalignment notification based at least partially on the classified characteristic of misalignment.

### BRIEF DESCRIPTION OF THE DRAWINGS

Many aspects of the present disclosure can be better understood with reference to the following drawings. The components in the drawing are not necessarily to scale. Instead, emphasis is placed on illustrating clearly the principles of the present disclosure. The drawings should not be taken to limit the disclosure to the specific embodiments depicted but are for explanation and understanding only.

FIG. 1 is a schematic view of an implementation of a video-based patient monitoring system configured in accordance with various embodiments of the present technology.

FIG. 2 is a block diagram illustrating an implementation of a video-based patient monitoring system having a computing device, a server, and one or more image-capturing devices and configured in accordance with various embodiments of the present technology.

FIG. 3 is a display view of an implementation of a user interface of a video-based patient monitoring system configured in accordance with various embodiments of the present technology.

FIG. 4 is a top view of implementations of alignments of a patient relative to a sensor.

FIG. 5 is a display view of an implementation of a alignment indicator system presentable on a sensor device or on a display.

FIG. 6 is a flow chart of an implementation of a method for qualifying sensor alignment relative to a patient environment configured in accordance with various embodiments of the present technology.

### DETAILED DESCRIPTION

The present disclosure relates to informative displays for non-contact patient monitoring. The technology described herein can be incorporated into systems and methods for non-contact patient monitoring. As described in greater detail below, the described technology can include obtaining respiratory data, such as via non-contact patient monitoring using image sensors (e.g., depth-sensing cameras), and displaying the respiratory data. The technology may further include comparing captured image data of a predefined portion of a patient and/or generated distance data relative to a predefined portion of a patient or an ROI to determine and/or classify whether and/or how a sensor is misaligned relative to the patient or ROI. The technology may further provide an instruction for a display representing the misalignment and/or potential corrective actions to respond to the misalignment. The instruction may include displaying an indication of the misalignment or classification of the misalignment on a display or an indicator physically located on the sensor.

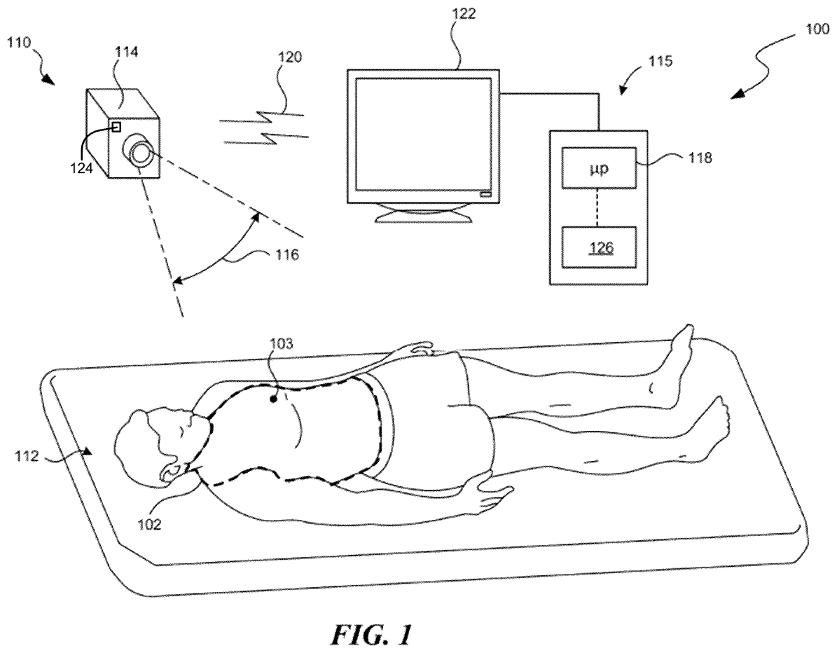

Specific details of several embodiments of the present technology are described herein with reference to FIGS. 1-6. Although many of the embodiments are described with respect to devices, systems, and methods for image-based monitoring of breathing in a human patient and associated display of this monitoring, other applications and other embodiments in addition to those described herein are within the scope of the present technology. For example, at least some embodiments of the present technology can be useful for image-based monitoring of breathing in other animals and/or in non-patients (e.g., elderly or neonatal individuals within their homes). It should be noted that other embodiments, in addition to those disclosed herein, are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a schematic view of a patient 112 and an implementation of a video-based patient monitoring system 100 configured in accordance with various embodiments of the present technology. The system 100 includes a non-contact detector 110 and a computing device 115. In some embodiments, the non-contact detector 110 can include one or more image capture devices, such as one or more video cameras. In the illustrated embodiment, the non-contact detector 110 includes the camera 114 (which may be a video camera adapted to capture video or other time-series image data). The non-contact detector 110 of the system 100 is placed remote from the patient 112. More specifically, a sensor or camera 114 of the non-contact detector 110 is positioned remotely from the patient 112 in that it is spaced apart from and does not contact the patient 112. The camera 114 includes a detector exposed to a field of view (FOV) 116 that encompasses at least a portion of the patient 112. In implementations, the camera 114 is operable to detect electromagnetic energy of any spectrum or other energy (e.g., infrared, visible light, thermal, x-ray, microwave, radio, gamma-ray, and the like).

The camera 114 can capture a sequence of images over time. The camera 114 can be a depth-sensing camera, such as a Kinect camera from Microsoft Corp. (Redmond, Washington) or Intel camera, such as the D415, D435, and SR305 cameras from Intel Corp. (Santa Clara, California). A depth-sensing camera can detect a distance between the camera and objects within its field of view. Such information can be used to determine that a patient 112 is within the FOV 116 of the camera 114 and/or to determine one or more regions of interest (ROI) to monitor on the patient 112. Once an ROI 102 is identified, the ROI 102 can be monitored over time, and the changes in depth of regions and/or captured images within the ROI 102 can represent movements of the patient 112 associated with breathing.

As described in greater detail in U.S. Patent Application Publication No. 2019/0209046, those movements, or changes of regions within the ROI 102, can additionally or alternatively be used to determine various breathing parameters, such as tidal volume, minute volume, respiratory rate, respiratory volume, etc. Those movements, or changes of regions within the ROI 102, can also be used to detect various breathing abnormalities, as discussed in greater detail in U.S. Patent Application Publication No. 2020/0046302. The various breathing abnormalities can include, for example, low flow, apnea, rapid breathing (tachypnea), slow breathing, intermittent or irregular breathing, shallow breathing, obstructed and/or impaired breathing, and others. U.S. Patent Application Publication Nos. 2019/0209046 and 2020/0046302 are incorporated herein by reference in their entirety.

In some embodiments, the system 100 determines a skeleton-like outline of the patient 112 to identify a point or points from which to extrapolate an ROI. For example, a skeleton-like outline can be used to find a center point of a chest, shoulder points, waist points, and/or any other points on the body of the patient 112. These points can be used to determine one or more ROIs. For example, an ROI 102 can be defined by filling in the area around a point 103, such as a center point of the chest, as shown in FIG. 1. Certain determined points can define an outer edge of the ROI 102, such as shoulder points. In other embodiments, instead of using a skeleton, other points are used to establish an ROI. For example, a face can be recognized, and a chest area inferred in proportion and spatial relation to the face. In other embodiments, a reference point of a patient's chest can be obtained (e.g., through a previous 3-D scan of the patient), and the reference point can be registered with a current 3-D scan of the patient. In these and other embodiments, the system 100 can define an ROI around a point using parts of the patient 112 that are within a range of depths from the camera 114. In other words, once the system 100 determines a point from which to extrapolate an ROI, the system 100 can utilize depth information from the camera 114 (which may be a depth-sensing camera) to fill out the ROI. For example, if the point 103 on the chest is selected, parts of the patient 112 around the point 103 that are a similar depth or distance from the camera 114 as the point 103 are used to determine the ROI 102.

In another example, the patient 112 can wear specially configured clothing or be covered with specifically configured bedding or other material (not shown) responsive to visible light or responsive to electromagnetic energy of a different spectrum that includes one or more features to indicate points on the body of the patient 112, such as the patient's shoulders and/or the center of the patient's chest. The one or more features can include a visually encoded message (e.g., bar code, QR code, etc.), and/or brightly colored shapes that contrast with the rest of the patient's clothing. In these and other embodiments, the one or more features can include one or more sensors that are operable to indicate their positions by transmitting light or other information to the camera 114. In these and still other embodiments, the one or more features can include a grid or another identifiable pattern to aid the system 100 in recognizing the patient 112 and/or the patient's movement. In some embodiments, the one or more features can be stuck on the clothing using a fastening mechanism such as adhesive, a pin, etc. For example, a small sticker can be placed on a patient's shoulders and/or on the center of the patient's chest that can be easily identified within an image captured by the camera 114. The system 100 can recognize the one or more features on the patient's clothing to identify specific points on the body of the patient 112. In turn, the system 100 can use these points to recognize the patient 112 and/or to define an ROI 102.

In some embodiments, the system 100 can receive user input to identify a starting point for defining an ROI 102. For example, an image can be reproduced on a display 122 of the system 100, allowing a user of the system 100 to select a patient 112 for monitoring (which can be helpful where multiple objects are within the FOV 116 of the camera 114) and/or allowing the user to select a point on the patient 112 from which an ROI can be determined (such as the point 103 on the chest of the patient 112). In other embodiments, other methods for identifying a patient 112, identifying points on the patient 112, and/or defining one or more ROI's can be used.

In an implementation, an ROI 102 may include a respiratory region of the patient 112. The respiratory region is a predefined region of a patient 112, the motion of which is attributable to breathing (e.g., chest, face, nostril, etc.). For example, a chest of the patient 112 typically moves during respiration. The chest cavity is moved to cause the lungs to inflate. The respiratory motion typically presents as consistent alternating chest expansion and relaxation. Other ROIs can include hands, faces or other anatomical features of patients. The respiratory region may be displayed on the display 122 as a regional indicator or “mask” over an image of the patient 112 and/or patient environment. The respiratory region and the regional indicator may be determined as described in U.S. patent application Ser. No. 16/713,265, which is incorporated herein by reference.

Detection of the ROI 102 can help determine whether a camera 114 (or another sensor) is aligned correctly with the patient environment. Types of misalignment between the camera 114 (or another sensor) and the ROI or other portion of the patient environment can include one or more of a direction of misalignment, a dimension of misalignment, a rotational misalignment, a translational misalignment, an angle of inclination misalignment, and a distance misalignment. A rotational misalignment may include that an azimuthal angle of the camera 114 or other sensor is misaligned, and an azimuthal angle correction can be used to correct the alignment. In an implementation, the system 100 may be configured to detect a rotational misalignment by detecting and determining the relative orientation or two or more of a patient's face, a patient's chest, a patient's hand, a visual indicator representing all or part of the ROI 102, and elements that cover (e.g., a blanket, face mask, or clothing) an anatomical feature of the patient. The rotational misalignment may also be based on a relative orientation of a longitudinal length of an ROI (e.g., that a central longitudinal axis of the ROI 102 should substantially bisect an FOV). A translational misalignment is one where a camera 114 is translated incorrectly relative to the patient environment such that a translational alignment correction can correct the misalignment. For example, a correct translational alignment may include that a center of an ROI 102 is centered in the FOV of the camera 114 (e.g., when the camera's 114 lens or detector is positioned substantially orthogonally relative to a captured surface (e.g., the ROI 102, a bed in which the patient lies, or a floor in a patient environment). An angle of inclination misalignment is an angular alignment of the camera's 114 lens (or other sensor detection element such as a depth sensor) relative to the ROI 102 that can be corrected by an angle of inclination correction. In an implementation, an aligned angle of inclination may be an angle of zero, indicating that the lens or other detection element detects light or electromagnetic radiation substantially (e.g., on average) orthogonally relative to a portion of the patient environment such as the ROI 102 or that the lens itself is substantially (e.g., on average) parallel with the surface of the ROI 102. The misalignment may be a distance misalignment such that a distance between the camera 114 is outside of a predefined range of distances. In implementations, the ROI 102 represents a respiratory region of the patient 112 or a portion of the patient environment the motion of which indicates respiration. In other implementations, a respiratory region separate of and/or adjacent to the ROI 102 can be determined in a manner similar to the manner in which the ROI 102 is determined. For example, implementations are contemplated in which the ROI 102 is a region over the chest area of the patient 112, and the determined respiratory region is a region over the face of the patient 112 that is substantially adjacent or near to the chest region.

The camera 114 can be used to generate sensor data representing one or more of images of the ROI or distances between points in the ROI and the camera 114 to distinguish patient 112 respiratory motion (at least largely) attributable to respiration from non-respiratory motion attributable to patient 112 actions other than respiration. The sensor data generated by the camera 114, including captured images and/or signals representing distances from points within the ROI and the camera 114, can be sent to the computing device 115 through a wired or wireless connection 120. The computing device 115 can include a hardware processor 118 (e.g., a microprocessor), the display 122, and/or hardware memory 126 for storing software and computer instructions. Sequential image frames of the patients and/or distance signals representing distances from the patient 112 are recorded by the camera 114 and sent to the hardware processor 118 for analysis. The analysis may be conducted by a signal processor and/or an alignment manager executable by the hardware processor 118. The analysis may include determinations of whether the camera 114 or other sensor is aligned relative to the patient environment. In implementations, the camera 114 includes an indicator that provides indications of alignment or misalignment of the camera 114 relative to the patient environment. In some embodiments, the display 122 can additionally or alternatively provide indications of alignment or misalignment whether the camera 114 is aligned or misaligned. Indications of misalignment may indicate one or more types of misalignment, one or more directions of misalignment, one or more magnitudes of misalignment (e.g., different magnitudes of different types of misalignment), and/or simply that the camera 114 is misaligned.

In an implementation, the camera 114 (or another sensor device) includes an alignment indicator 124. In the illustrated implementation, the alignment indicator 124 is an indicator light that can emit light differently depending on whether the camera 114 is aligned relative to the patient 112. For example, in an implementation, the alignment indicator 124 emits a green light when the camera 114 is appropriately aligned and emits a red light when the camera 114 is misaligned. The camera may have more complex light arrangements in the alignment indicator 124, such as lights that specifically indicate one or more of types, directions, and magnitudes of misalignments, lights that indicate corrective actions for one or more of types, directions, and magnitudes of misalignments, and an active pixelated display to display one or more of the type, directions, and magnitudes of misalignments and corrective actions. Displaying the classifications and/or corrective actions on the alignment indicator 124 of the camera 114 may help a user align the camera 114 without having to look at a separate display (e.g., display 122), which may be located in a different room. In implementations, the instructions from an alignment manager may additionally or alternatively cause the misalignment classifications and/or corrective actions to be displayed on display 122.

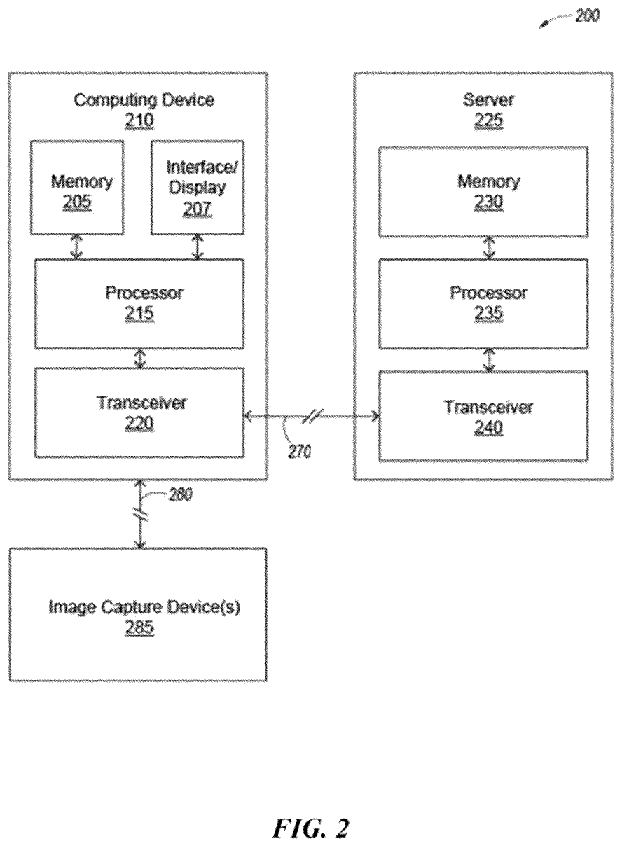

The display 122 can be remote from the camera 114, such as a video screen positioned separately from the hardware processor 118 and the hardware memory 126. Other embodiments of the computing device 115 can have different, fewer, or additional components than shown in FIG. 1. In some embodiments, the computing device 115 can be a server. In other embodiments, the computing device 115 of FIG. 1 can be additionally connected to a server (e.g., as shown in FIG. 2 and discussed in greater detail below). The captured images/video can be processed or analyzed by the signal processor at the computing device 115 and/or a server to determine a variety of parameters (e.g., respiratory patient motion, non-respiratory patient motion, tidal volume, minute volume, respiratory rate, etc.) of the patient's breathing. In some embodiments, some or all of the processing may be performed by the camera, such as by a hardware processor integrated into the camera or when some or all of the computing device 115 is incorporated into the camera.

FIG. 2 is a block diagram illustrating an implementation of a video-based patient monitoring system 200 (e.g., the video-based patient monitoring system 100 shown in FIG. 1) having a computing device 210 (e.g., an implementation of the computing device 115), a server 225, and one or more image capture device(s) 285, and configured in accordance with various embodiments of the present technology. In various embodiments, fewer, additional, and/or different components can be used in the system 200. The computing device 210 includes a hardware processor 215 (e.g., an implementation of the hardware processor 118) that is coupled to a memory 205. The hardware processor 215 can store and recall data and applications in the memory 205, including applications that process information and send commands/signals according to any of the methods disclosed herein. The hardware processor 215 can also (i) display objects, applications, data, etc. on an interface/display 207 and/or (ii) receive inputs through the interface/display 207. As shown, the hardware processor 215 is also coupled to a transceiver 220.

The computing device 210 can communicate with other devices, such as the server 225 and/or the image capture device(s) 285 via (e.g., wired or wireless) connections 270 and/or 280, respectively. For example, the computing device 210 can send to the server 225 information determined about a patient from images captured by the image capture device(s) 285. The computing device 210 can be the computing device 115 of FIG. 1. Accordingly, the computing device 210 can be located remotely from the image capture device(s) 285, or it can be local and close to the image capture device(s) 285 (e.g., in the same room). In various embodiments disclosed herein, the hardware processor 215 of the computing device 210 can perform the steps disclosed herein. In other embodiments, the steps can be performed on a hardware processor 235 of the server 225. The hardware processor 235 of the server 225 is coupled to a memory 230. The hardware processor 235 can store and recall data and applications in the memory 230. The hardware processor 235 is also coupled to a transceiver 240. In some embodiments, the hardware processor 235, and subsequently the server 225, can communicate with other devices, such as the computing device 210, through a connection 270.

In some embodiments, the various steps and methods disclosed herein can be performed by both of the hardware processors 215 and 235. In some embodiments, certain steps can be performed by the hardware processor 215 while others are performed by the hardware processor 235. In some embodiments, information determined by the hardware processor 215 can be sent to the server 225 for storage and/or further processing.

In an implementation, the image capture device(s) 285 generate sensor data such as captured images and/or signals representing distances between the image capture device(s) 285 and at least one point in an ROI. In some embodiments, the image capture device(s) 285 are remote sensing device(s), such as depth-sensing video camera(s), as described above with respect to FIG. 1.

In some embodiments, the image capture device(s) 285 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter-wave scanning technology can be utilized to scan a patient, which can be used to define and/or monitor an ROI. Advantageously, such technologies can be able to penetrate (e.g., “see”) through clothing, bedding, or other materials while giving an accurate representation of the patient's skin. This can allow for more accurate measurements, particularly if the patient is wearing baggy clothing or is under bedding. The image capture device(s) 285 can be described as local because they are relatively close in proximity to a patient such that at least a part of a patient is within the field of view of the image capture device(s) 285.

In some embodiments, the image capture device(s) 285 can be adjustable to ensure that the patient is captured in the field of view. For example, the image capture device(s) 285 can be physically movable, can have a changeable orientation (such as by rotating or panning), and/or can be capable of changing a focus, zoom, or other capture characteristic to allow the image capture device(s) 285 to adequately capture images of a patient and/or an ROI of the patient. In various embodiments, for example, the image capture device(s) 285 can focus on an ROI, zoom in on the ROI, center the ROI within a field of view by moving the image capture device(s) 285, or otherwise adjust the field of view to allow for better and/or more accurate tracking/measurement of the ROI. The system 200 may include automatic actuators to align the image capture device(s) 285 based on a determination that the image capture device(s) are misaligned. The corrective measures may include one or more of a distance (between the image capture device(s) and the ROI) correction, an azimuthal angle (rotational) correction, a translational correction, and an angle of inclination correction. In implementations, indicator lights may also function as buttons to drive the actuator to correct the misalignment by indicated types of corrective measures to correct types, directions, and/or magnitudes of misalignment.

In an implementation, the generated sensor data can include time-series data. For example, the sensor data can be arranged chronologically, perhaps with associated timestamps representing data capture and/or generation times. The time-series data may represent patient motion over time. The time-series data can represent video data for captured images and can represent changes in distances between the image capture device(s) 285 and points in the ROI for distance signal data. The time-series data can be analyzed to show misalignment over time and/or changes in misalignment over time and can be used to determine misalignment. When time-series data is analyzed, durations including one or more time windows (e.g., between a first time and a second time) and/or sample sizes may be used during the analysis. The durations may be dynamically determined based on the breathing patterns of a particular patient or may be standardized.

The system 200 may include an alignment manager for processing the sensor data generated by the image capture device(s) 285 and determining whether the image capture device(s) 285 are appropriately aligned relative to a patient or an element of the patient's environment (e.g., an ROI, a respiratory region, or identifiable element near an ROI). The alignment manager may include a hardware element of one or more of the computing device 210, the image capture device(s) 285, and the server 225, may include a software element executable by one or more of a processor of the image capture device(s) 285, the hardware processor 215, and the hardware processor 235, or may include a hybrid system of software and hardware contained in one or more of the computing device 210, the image capture device(s) 285, and the server 225.

In some embodiments, the alignment manager generates an aggregate alignment metric of the alignment between the image capture device(s) 285 and the patient environment. In these embodiments, the alignment manager receives the generated sensor data (e.g., captured images of and/or sensed distances from an element of a patient environment, such as a respiratory region of a patient) and is operable to generate, based at least in part on the detected sensor data, an aggregate alignment metric. The aggregate alignment metric may include an aggregate distance or angular metric based on one or more of a mean, a medial, and a trimmed mean of detected distances between the image capture device(s) 285 and the element of the patient environment. The aggregate alignment metric may account for multidimensional misalignments of different misalignment types, such as one or more of a rotational misalignment, a translational misalignment, an angle of inclination misalignment, and a distance misalignment. The aggregate alignment metric may further account for the magnitude and/or direction of the types of misalignment. The alignment manager may then determine whether the aggregate alignment metric satisfies a misalignment condition. The misalignment condition may include a predefined threshold value and/or predefined range of values representing magnitudes and/or directions for the misalignment types. In implementations, the aggregate alignment metric may include a score that is based on values of the misalignment types. If the score is multifactorial (e.g., based on values of more than one alignment type or based on different methods of determining a nature or extent of the alignment), the score may include a weighted average of each of the different factors.

Implementations of aggregate alignment metrics may include alignment scores. In one implementation, the alignment score is determined based on an aggregate distance between the image capture device(s) 285 and the determined respiratory region. For example, the alignment manager may be able to determine an alignment score as in equation 1.

$$alignment score = \vert {distance}_{agg} - 1 \vert \qquad ( 1 )$$

The aggregate distance (distance_{agg}) may include one or more of an average, a median, and a trimmed mean of sensed distance data. In equation one, an absolute value is taken of a meter subtracted from the aggregate distance. If the distance is too different from one meter, the alignment score will reflect that the image capture device(s) 285 are misaligned. Predefined distances other than one meter are contemplated and may be substitued into one or more of equation 1 and equation 2. In implementations, the alignment score will be further based on an aggregate angle, as presented in equation 2.

$$alignment score = \vert {distance}_{agg} - 1 \vert + \vert {angle}_{agg} \vert \qquad ( 2 )$$

The aggregate angle (angle_{agg}) may be based on one or more of an average, median, or mean of angles detected between the image capture device(s) 285 and the determined respiratory region or an angle representative of a mean or median vector of determined distances between the image capture device(s) 285 and an element of the patient environment. The misalignment conditions may be based on one or more of the alignment scores. In implementations, the misalignment condition may include that the misalignment condition is satisfied when the alignment scores exceed a threshold alignment score or fall outside of an acceptable range of alignment scores.

In embodiments, the alignment manager additionally or alternatively determines a sensed quality of the respiratory region. In these embodiments, the alignment manager receives detected sensor data (e.g., captured images of and/or sensed distances from an element of a patient environment, such as a respiratory region of a patient) and is configured to determine by a predefined relationship between sensor data and sensed qualities of a respiratory region, a sensed quality of the determined respiratory region based on the detected sensor data. The sensed quality may include one or more of a detected size or shape of the determined respiratory region, a detected fill ratio of an image representing the respiratory region (e.g., the extent to which there are gaps in a visual indicator overlaid over an image representing the respiratory region), and a detected shape or size of a respiratory mask (e.g., a region determined to be a respiratory region of a patient). The alignment manager may then determine whether the sensed quality satisfies a misalignment condition. For example, the sensed shape or size of the sensed portion of the determined respiratory region may differ depending upon the angle and/or distance from which the image capture devices 285 sense the respiratory region. If the sensed shape or size of the respiratory region is consistent with predefined shape and/or size parameters of a misalignment condition, the sensed quality of sensed shape or size may satisfy the misalignment condition.

In implementations in which a sensed size of the portion of the detected respiratory region is used to determine misalignment, the sensed size of the respiratory region relative to the size of a comparison region (e.g., one or more of the ROI, the whole of the respiratory region, a superimposed targeting portion imparted by the sensor or image processor to the sensor data when captured) may indicate a distance between the image capture device(s) 285 and the respiratory region. The ratio may be determined based on a number of pixels in the respiratory region relative to a number of pixels in the rest of the sensed environment (or within a predefined range of the field of view of the image capture device(s) 285) or the relative size may be determined by integrating the depth or distance in the respiratory region or another indicated region (e.g., a region covering one or more of a patient's chest, head, arm, hand, other anatomical feature that can demonstrate orientation of the image capture device(s) 285 relative to the patient) to generate a physical measure of the size of the indicated region.

In implementations in which the sensed shape is of a chest of a respiratory region of the patient, the sensed shape may be expected to be substantially elliptical and substantially symmetrical about a bisector of the elliptical shape. Dissimilarity in size and or shape (e.g., as elements of a misalignment condition) may be used to determine misalignment of the image capture device(s) 285.

Additionally or alternatively, if a sensed fill ratio (ratio of distances to positions in the determined respiratory region detected that fail to satisfy an illuminating condition to present as light in a respiratory region or mask based on absolute distances to positions in the determined respiratory region or relative to other detected distances in the determined respiratory region that satisfy the illuminating condition) is an element of a misalignment condition, if the ratio falls within a predetermined range or satisfies a predetermined threshold of the misalignment condition, the sensed quality of fill ratio may satisfy the misalignment condition.

In implementations, the predefined relationship between the generated sensor data and the sensed quality used to determine satisfaction of the misalignment condition can be represented in an inferential model such as a machine learning model. The inferential model may be pre-trained (e.g., before inferential determinations are made) based on labeled generated sensor data that is labeled as representing a sensed quality of a respiratory region. For example, the inferential model may be pre-trained by inputting received time-series data of captured images and/or generated distance signals at different times (e.g., a first time and a second time) that are labeled with the labeled sensed quality of a determined respiratory region. An inferential model trainer may then compare the output of the inferential model with the label associated with the input and determine a loss between the output and the associated label. The inferential model trainer may then backpropagate or otherwise distribute the loss through the inferential model (e.g., by adjusting one or more of weights, activation functions, and biases represented in one or more of nodes and edges of a neural network of a machine learning model). By repeating this process with different labeled generated sensor data, the inferential model can represent the predefined relationship between generated sensor data and sensed qualities.

In implementations, the labels and output of the inferential model can include data representing confidence in the output determination (e.g., probabilistic and/or statistical data). In implementations, a predefined relationship including an inferential model may similarly be applied between the detected sensor data and one or more aggregate alignment metrics to determine aggregate alignment metrics based on the detected sensor data which may be pre-trained analogously using sensor data labeled with labeled aggregate alignment metrics. Further still, implementations are considered where a single inferential relationship can determine, based on detected sensor data, one or more sensed qualities, and one or more aggregate alignment metrics representing alignment between the image capture device(s) and the determined respiratory region, a classification or score of misalignment.

In implementations, the inferential model may include, without limitation, one or more of data mining algorithms, artificial intelligence algorithms, masked learning models, natural language processing models, neural networks, artificial neural networks, perceptrons, feedforward networks, radial basis neural networks, deep feedforward neural networks, recurrent neural networks, long/short term memory networks, gated recurrent neural networks, autoencoders, variational autoencoders, denoising autoencoders, sparse autoencoders, Bayesian networks, regression models, decision trees, Markov chains, Hopfield networks, Boltzmann machines, restricted Boltzmann machines, deep belief networks, deep convolutional networks, genetic algorithms, deconvolutional neural networks, deep convolutional inverse graphics networks, generative adversarial networks, liquid state machines, extreme learning machines, echo state networks, deep residual networks, Kohonen networks, support vector machines, federated learning models, and neural Turing machines.

In implementations, the inferential model may be trained by an inferential model trainer using a training method. Examples of training methods (e.g., inferential and/or machine learning methods) can include, without limitation, one or more of masked learning modeling, unsupervised learning, supervised learning, reinforcement learning, self-learning, feature learning, sparse dictionary learning, anomaly detection, robot learning, association rule learning, manifold learning, dimensionality reduction, bidirectional transformation, unidirectional transformation, gradient descent, autoregression, autoencoding, permutation language modeling, two-stream self attenuation, federated learning, and the like.

The predefined relationship may be based on the demographics of the patient. In implementations, the predefined relationship is made specifically for the demographic of the patient by training the inferential model exclusively or predominantly on labeled data associated with the demographic. In another implementation, the inferential model is configured and/or pre-trained to take the demographic data as input, and the inferential relationships within the inferential model account for the demographic data internally.

The system can additionally or alternatively determine other respiratory measurements such as values of a variety of parameters (e.g., respiratory patient motion, non-respiratory patient motion, tidal volume, minute volume, respiratory rate, etc.). In implementations, the signal processor can determine the respiratory measurements based on the generated sensor data. In other implementations, the signal processor can receive respiratory measurements generated by other means, including one or more of a transthoracic impedance measurement, an electrocardiogram, capnograph, spirometer, pulse oximeter, and a manual user entry. Implementations are also contemplated in which the signal processor does not process respiratory measurements other than the classification of patient motion. In implementations, the respiratory measurements may be accounted for in one or more of the determining of the aggregate alignment metric, determining the sensed quality, and the misalignment condition. For example, the misalignment condition may be based on a periodicity of a respiratory volume waveform. Irregular periodicity may be indicative of a misalignment, and regular waveform periodicity may be indicative of correct alignment. Similarly, respiratory rate measurements and/or associated confidence values for the respiratory rate measurements, if they are satisfactorily dissimilar to expected or predefined respiratory rate measurements (e.g., in satisfaction of a dissimilarity condition), may indicate that the image capture device(s) 285 are misaligned relative to the patient environment.

The alignment manager, upon determination of one or more alignment qualities (e.g., aggregate alignment metric and/or sensed quality), may then determine, based on the one or more alignment qualities, whether the one or more alignment qualities satisfy one or more misalignment conditions. In implementations, a misalignment condition may include a predefined threshold value and/or predefined range of values representing magnitudes and/or directions for the misalignment types. In implementations, the aggregate alignment metric may include a score that is based on values of the misalignment types. If the score is multifactorial (e.g., based on values of more than one alignment type or based on different methods of determining a nature or extent of the alignment), the score may include a weighted average or geometric mean of each of the different factors.

In an implementation, a misalignment condition may be at least partially based on a relative orientation of a region near an ROI. For example, the determined respiratory region can include an ROI of a chest region and one or more anatomical features, including, without limitation, a determined adjacent head, arm, leg, or hand. If the head is not directly above the torso in an appropriate dimension, it may indicate that the image capture device(s) 285 are rotationally misaligned (misaligned azimuthally).

In other implementations, the misalignment conditions may include hard cutoff values for certain parameters. For example, even if the determination of satisfaction of one or more misalignment conditions is based on many factors, one or more factors can have threshold values or ranges in which the misalignment condition will automatically fail. For example, a misalignment condition may include a maximum aggregate distance of 1.5 meters between the image capture device(s) 285 and the respiratory region. In this example, even if all other values indicate that the image capture device(s) 285 are aligned, the misalignment condition will be satisfied solely based on the determination that the aggregate distance is 1.6 meters, ignoring all other factors. In this implementation, detecting that the determined respiratory region is more than 1.5 meters away indicates a misalignment regardless of the other factors considered.

In implementations in which the alignment manager makes determinations of one or more of aggregate alignment metrics, sensed qualities, satisfaction of misalignment conditions (or the misalignment conditions the alignment manager uses to determine the satisfaction), and misalignment classifications based on more than one of the aforementioned factors, the factors may be weighted and considered in a weighted sum, or the factors may be used to determine a geometric mean as an overall misalignment score or a score for particular misalignment elements (e.g., misalignment types, directions, and/or magnitudes).

In implementations, the alignment manager may classify the one or more alignment qualities. In these implementations, the alignment manager may include a misalignment classifier operable to classify a characteristic of the misalignment based on the satisfaction of the misalignment condition. The classified characteristic of misalignment may include one or more of that the image capture device(s) 285 are misaligned relative to the determined respiratory region, one or more types of misalignment (e.g., rotational, translational, incline angle, and/or distance), and one or more magnitudes (e.g., of one or more types) of misalignment. Implementations are contemplated in which the classification is performed by a same inferential relationship as or a different inferential relationship from the inferential relationship that determines one or more of the aggregate alignment metric and/or the sensed quality of the determined respiratory region. In implementations that use the same inferential relationship, the operations of determining the sensed quality and/or aggregate metric and the operation of classification of a characteristic of misalignment may be united into a single operation (e.g., the input is sensor data, and the output is a classification of misalignment).

In implementations, the alignment manager or an instruction generator generates an instruction to provide a misalignment notification based at least in part on the satisfaction of the misalignment condition and/or a classification of the misalignment (e.g., determined based on the satisfaction of the misalignment condition). The alignment manager and/or the instruction generator of the alignment manager executable by a hardware processor (e.g., one or more of hardware processor 215, hardware processor 235, or a hardware processor of the image capture device(s) 285) of the system 200 can generate instructions for the display of data generated by the alignment manager. The instructions can include data representing an instruction to display a misalignment notification. The misalignment notification may include one or more of an indication that the image capture device(s) 285 and the determined respiratory region are misaligned, a type of misalignment, a magnitude of misalignment, and one or more corrective actions to take in order to align the image capture device(s) relative to the determined respiratory region.

In implementations, the instruction and/or indication may include data representing one or more of a motion classification flag, a classification-specific display, an overlaid display (e.g., configured to overlay a displayed element in a user interface to indicate a misalignment relative to the determined respiratory region), an image representation of patient motion (e.g., a visual or video representation of captured images and/or generated distance signals), an audio signal, a flashing element (e.g., where the magnitude of light of elements of a display is alternatively increased and decreased), a different alert, a code representing the aforementioned items, and the like. In implementations in which the classification is one of a number of classifications, each classification may correspond to a different display. The signal processor may output data representing the motion classification or a specific display associated in memory of the system 200 with the alignment classification. For example, different classifications can be represented in a display by different colors, different magnitudes of light in the display, different frequencies at which to flash light in the display, and the like.

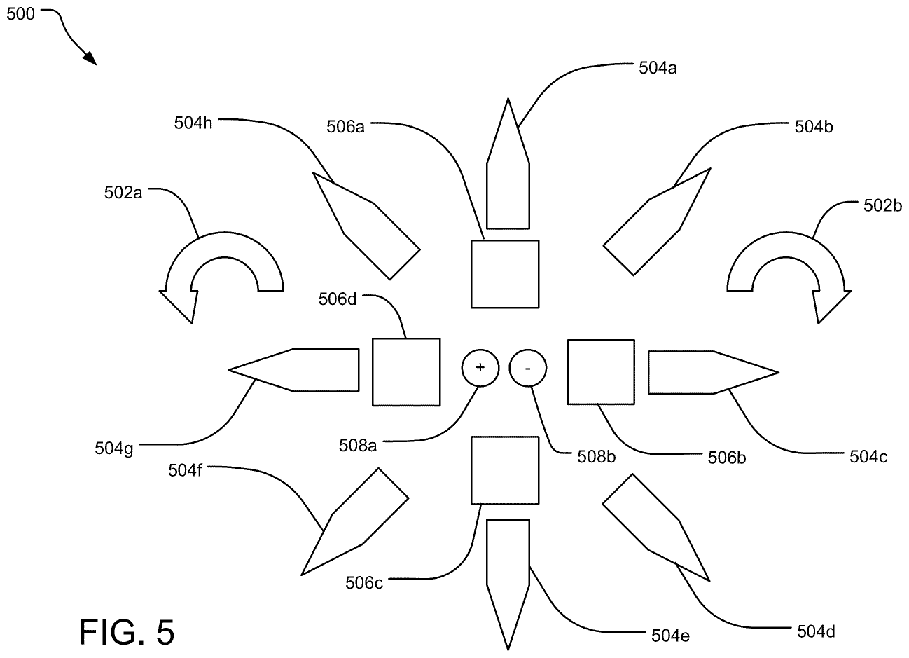

In implementations in which the misalignment classifications are representative of different degrees of misalignment or confidence in measurements thereof, the corresponding display may represent a spectrum or range of color, light magnitude, or flash frequency based on a magnitude of the one or more of the different degrees of the types of misalignment and/or different degrees of confidence in the determination of the misalignments. In one implementation, the instruction includes activating one or more lights on the image capture device(s) 285 (e.g., an implementation is illustrated as alignment indicator 124 of the camera 114 in FIG. 1). The light display may be simple and indicate that there is a misalignment, whether by emitting different colors of light, only activating the light when the image capture device(s) 285 are one of aligned or misaligned, only flashing a light when the image capture device(s) 285 are one of aligned or misaligned, and the like. The display may be more complex in that it indicates one or more of a type and magnitude of misalignment and a type and magnitude of a corrective action to correct the misalignment. For examples of different indicator lights, see the alignment indicator system 500 of FIG. 5. In implementations, the magnitude of any particular type of misalignment can be represented by a frequency at which, a color of, and/or a brightness of intensity of the lights or pixels that represent a particular type or direction of misalignment or represent a particular type or direction of a corrective action to correct the misalignment.

In implementations, the instruction to provide a misalignment notification may be configured to cause a display to display the misalignment notification as an element overlaid over or underlaid under (e.g., displayed behind) another displayed respiratory measurement or an image of the patient, including an ROI image to indicate the alignment of the image capture device(s) 285. For example, the display may be configured to display a measured respiratory rate and may display the indication of misalignment classification over or under the displayed respiratory rate. An overlaid or underlaid display of the misalignment notification may be configured to be visually contrasted from the displayed respiratory measurement. For example, the elements may be of different colors and/or may be of different transparencies, and/or the displayed misalignment notification may be at least partially transparent to maintain visibility of the displayed respiratory measurement (e.g., appearing as a highlighting of or patch over the displayed respiratory measurement). Additionally or alternatively, the displayed misalignment notification may be overlaid over an ROI image. In an alternative implementation, the instruction instructs a display not to display the respiratory measurements and/or patient images when the alignment manager determines that the image capture device(s) 285 are misaligned.

In implementations, the misalignment manager may be communicatively coupled with a patient presence detector (e.g., hardware or software executable by one or more hardware processors) that detects whether a patient is present. In implementations in which the presence detector determines that no patient is present, the misalignment manager may one or more of deactivate itself (e.g., until a patient's presence is detected), deactivate any display or indication the misalignment manager would instruct to display an indication of misalignment, and transmit an instruction to display a different indication of alignment (e.g., color, light frequency, and/or intensity) reflecting that there is no patient present.

The devices shown in the illustrative embodiment can be utilized in various ways. For example, either of the connections 270 and 280 can be varied. Either of the connections 270 and 280 can be a hard-wired connection. A hard-wired connection can involve connecting the devices through a USB (universal serial bus) port, serial port, parallel port, or another type of wired connection that can facilitate the transfer of data and information between a processor of a device and a second processor of a second device. In another embodiment, either of the connections 270 and 280 can be a dock where one device can plug into another device. In other embodiments, either of the connections 270 and 280 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication can include near-field communications, such as passive radio-frequency identification (RFID) and active RFID technologies. RFID and similar near-field communications can allow the various devices to communicate over a short range when they are placed proximate to one another. In yet another embodiment, the various devices can connect through an internet (or another network) connection. That is, either of the connections 270 and 280 can represent several different computing devices and network components that allow the various devices to communicate through the internet, either through a hard-wired or wireless connection. Either of the connections 270 and 280 can also be a combination of several modes of connection.

The configuration of the devices in system 200 of FIG. 2 is merely one physical system on which the disclosed embodiments can be executed. Other configurations of the devices shown can exist to practice the disclosed embodiments. Further, configurations of additional or fewer devices than the devices shown in FIG. 2 can exist to practice the disclosed embodiments. Additionally, the devices shown in FIG. 2 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, sensors that detect other portions of the electromagnetic spectrum, night vision cameras/detectors, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

Referring back to FIG. 1, the display 122 can be used to display various information regarding the patient 112 monitored by the system 100. In some embodiments, the system 100, including the camera 114, the computing device 115, and the hardware processor 118, is used to generate sensor data (e.g., captured images and/or generated distance signals) and, by a signal processor, determine classifications of patient motion that can be displayed in a user interface presented on the display 122 or otherwise indicated as described with respect to system 200 of FIG. 2. Additionally or alternatively, the system 100, including the camera 114, the computing device 115, and the hardware processor 118, is used to generate, receive, and/or display respiratory measurement data (e.g., respiratory rate) in the same or a different user interface. Additionally or alternatively, the system 100, including the camera 114, the computing device 115, and the hardware processor 118, is used to generate, receive, and/or display the generated sensor data as an image of the patient and/or the ROI.

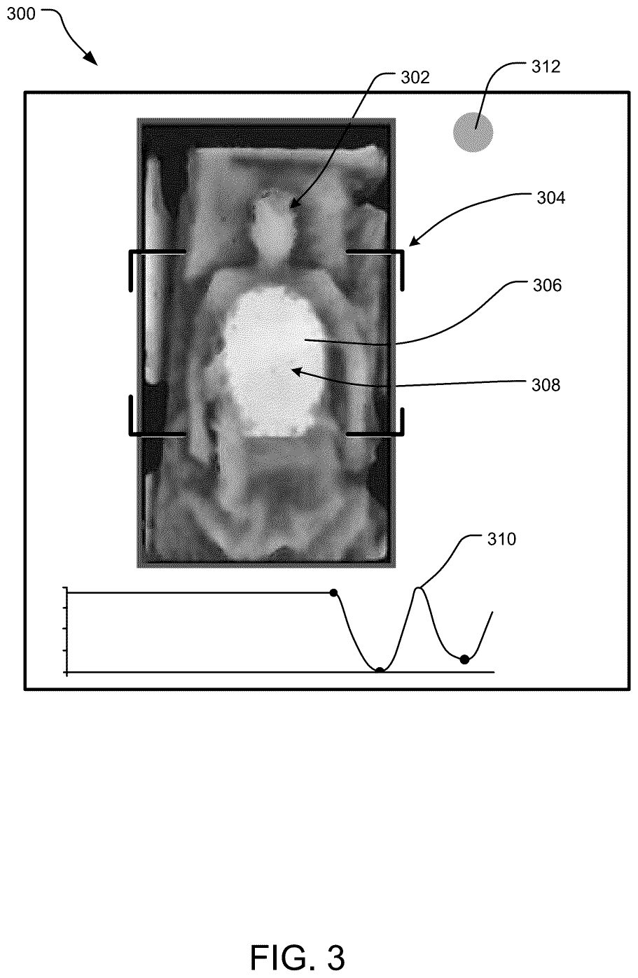

FIG. 3 is a display view of an implementation of a user interface 300 of a video-based patient monitoring system configured in accordance with various embodiments of the present technology. In implementations, the user interface 300 may include one or more of a visual representation of a patient 302, a superimposed targeting rectangle 304, an ROI 306 of the patient 302 (e.g., the patient's 302 chest), a visual indicator 308 representing a respiratory region of the patient 302, a measurement of respiratory function 310 (illustrated as a respiratory volume signal), and an alignment indicator 312. In the illustrated implementation, the user interface 300 includes the visual representation of the patient 302 and the ROI 306. In the illustrated user interface 300, the visual indicator 308 highlights a portion of the ROI 306 designated as the respiratory region. In implementations, the alignment indicator 312 may be used to indicate whether a sensor (e.g., camera or other image capture device) is aligned in a predefined manner that does not satisfy a misalignment condition. In the illustrated implementation, the alignment indicator 312 is merely a simple indicator that can indicate by color, pattern, frequency of flashing, or intensity (brightness) of light whether the sensor is aligned relative to the patient environment. Implementations are contemplated in which the same or different indicator is additionally or alternatively presented on a surface or light indicator on the sensor itself to aid in appropriately aligning the sensor relative to the patient environment (e.g., one or more of the ROI 306, the elements represented by the visual indicator 308, and the patient 302). The visual indicator 308 can be an element of any device or a standalone device that can allow a clinician to view the visual indicator 308 in a clinical environment and use the visual indicator to determine and/or correct misalignments of the sensors relative to the patient environment.

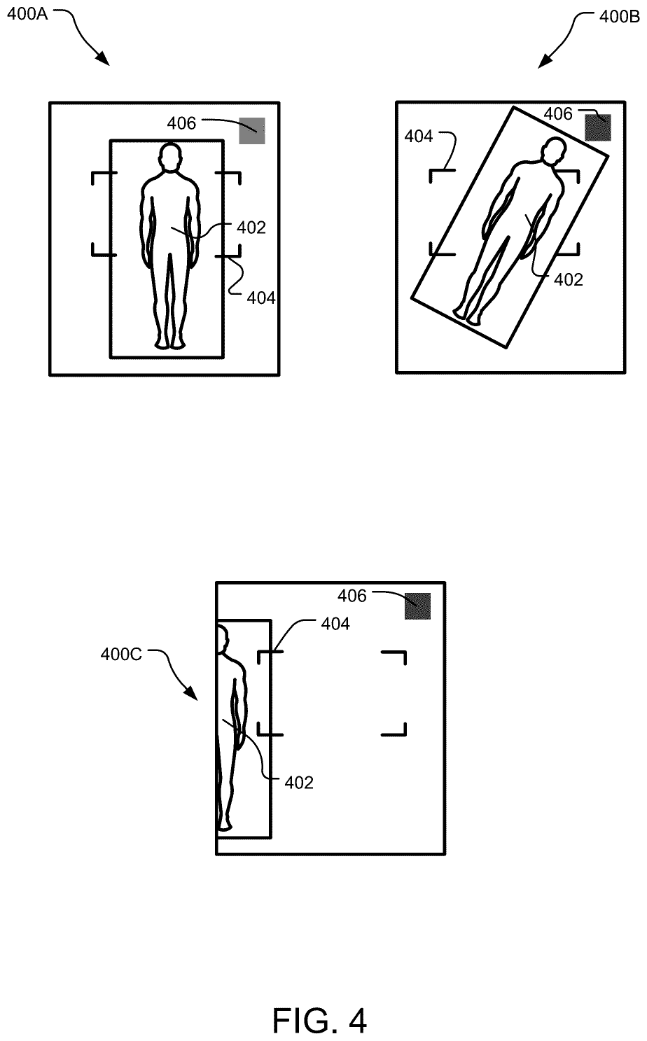

FIG. 4 is a top view of implementations of alignments 400A-400C of a patient 402 relative to a sensor. In a first alignment 400A, the sensor is aligned with the patient 402. A patient is centrally located in a superimposed targeting rectangle 404, and an axis bisecting the head and feet of the patient 402 substantially bisects the top and bottom boundaries of the superimposed targeting rectangle 404. This indicates that one or more of the distance between the sensor and the patient, the azimuthal (rotational) angle between the sensor and the patient 402, the angle of inclination between the sensor and the patient 402, and the translational location of the sensor relative to the patient 402 are aligned. Accordingly, the pattern of an alignment indicator 406 indicates that the sensor is aligned relative to the patient 402.

In a second alignment 400B, the patient 402 is rotated relative to the superimposed targeting rectangle 404. This indicates a rotational misalignment which a rotational (azimuthal) angle correction could correct. The system may detect (e.g., based on one or more of a predefined relationship, an aggregate alignment metric, and a sensed quality) that the rotation is misaligned and correspondingly indicates by alignment indicator 406 that the sensor is misaligned relative to the patient.

In a third alignment 400C, the sensor is translated relative to the patient 402. This can be determined based on the patient 402 being outside of (or otherwise not centered in) the superimposed targeting rectangle 404. This translational misalignment causes the alignment indicator 406 to indicate that the sensor is not aligned with the patient 402.

More complex alignment indicators 406 may indicate more than whether the sensor is aligned relative to the patient 402. For example, the indicators may indicate one or more types of misalignment, one or more magnitudes (e.g., of the one or more types of misalignment) of misalignment, and corrective actions to correct the misalignment (which may include type and/or magnitude data).

FIG. 5 is a display view of an implementation of an alignment indicator system 500 presentable on a sensor device, a display, a standalone device, or anywhere visible to a clinician in a clinical environment. The alignment indicator system 500 can include one or more of rotational (azimuthal) alignment indicators 502a, 502b, angle of inclination alignment indicators 504a-504h, translational alignment indicators 506a-d, and distance alignment indicators 508a, 508b.

In the illustrated implementation, the alignment indicators 502, 504, 506, and 508 may be presented to demonstrate the types of misalignment and, additionally or alternatively, types, directions, and/or magnitudes of alignment corrections to be applied. For example, if certain elements are illuminated, they may indicate a type, direction, and/or magnitude of a misalignment or a correction to correct a misalignment.

The rotational (azimuthal) alignment indicators 502a, 502b each specify a different direction of rotation and may indicate a rotational misalignment or a rotational misalignment correction. The angle of inclination alignment indicators 504a-504h each specify an angle of inclination misalignment or correction. For example, the sensor (e.g., a camera) may be attached by a pivot to an articulating arm. Movement of the sensor relative to the pivot without rotation will change the angle of inclination of the sensor relative to a patient. The angle of inclination alignment indicators 504a-504h can indicate a direction and magnitude of the misalignment or alignment correction. The translational alignment indicators 506a-d indicate translational misalignments or translational misalignment corrections. For example, the translational misalignment corrections may indicate how to manipulate (e.g., move translationally) an articulating arm to which the sensor is coupled to correct a translational misalignment. Distance alignment indicators 508a, 508b may indicate whether a distance between the sensor and the patient is incorrect (misaligned). In an implementation in which the distance alignment indicators 508a, 508b indicate misalignment corrections, emitting light from a first distance alignment indicator 508a may indicate that the distance between the sensor and the patient needs to be increased (by moving an articulating arm to which the sensor is coupled further away), and emitting light from a second distance alignment indicator 508b may indicate the opposite.

One or more of the alignment indicators 502, 504, 506, and 508 may also present magnitude information to represent a magnitude of misalignment or misalignment correction. The magnitude information may include a frequency of blinking of an emitted light of an indicator, an intensity or brightness of a light of an indicator, or a color of a light of an indicator (e.g., within a predefined representative scheme). In implementations, a guide may be presentable by the alignment indicators 502, 504, 506, and/or 508 (whether on the sensor or on a display) to allow a user to interpret the light emitted by the alignment indicators 502, 504, 506, and/or 508.

Implementations are contemplated in which multiple indicators emit light simultaneously to indicate that a misalignment or correction is somewhere between the indicators. For example, the translational alignment indicators 506a and 506b may both emit light to indicate that the translational misalignment is in a direction between them. Magnitude information may be used to indicate in which of the directions indicated by the translational alignment indicators 506a and 506b the sensor is more misaligned.

Implementations are contemplated in which a single light indicator may indicate that any type of alignment is incorrect and may present differently depending on the direction and magnitude of the misalignment or correction. For example, the indicator could be a single rotational alignment indicator that presents a color of a palette that indicates the one or more of magnitude and direction of the misalignment. In another implementation, the color could indicate the direction of misalignment, and a frequency of flashing of a light or an intensity of light could indicate the magnitude of the indicated type of misalignment.

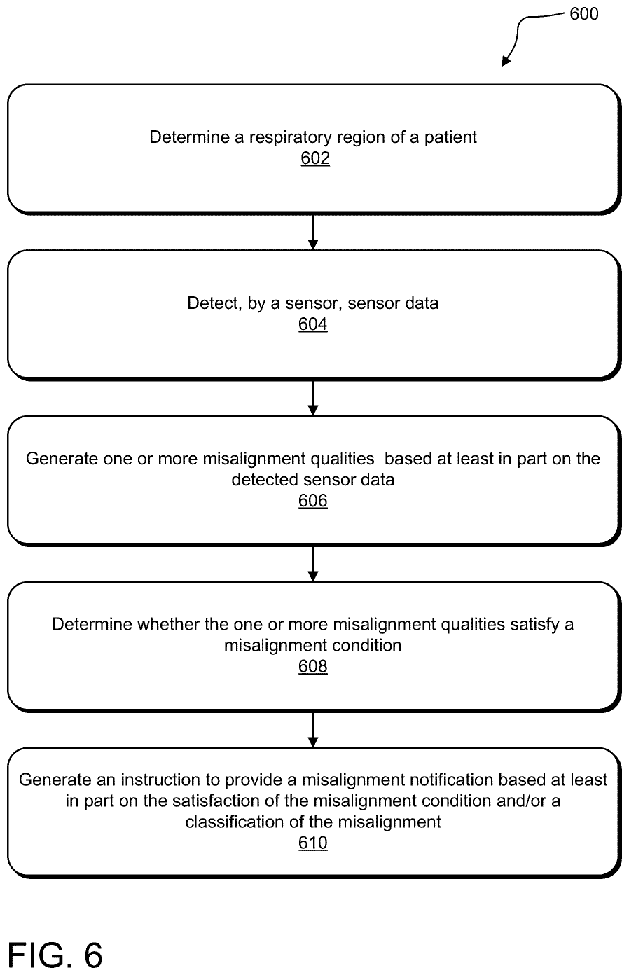

FIG. 6 is a flow chart of an implementation of a method 600 for qualifying sensor alignment relative to a patient environment configured in accordance with various embodiments of the present technology. In step 602, a respiratory region of the patient is determined. In an implementation, a region of interest (ROI) may include a respiratory region of the patient. The respiratory region is a predefined region of a patient, the motion of which is attributable to breathing (e.g., chest, face, nostril, etc.). For example, a chest of the patient typically moves during respiration. The chest cavity is moved to cause the lungs to inflate. The respiratory motion typically presents as consistent alternating chest expansion and relaxation. Other ROIs can include hands, faces, or other anatomical features of patients.

The respiratory region may be displayed on the display as a regional indicator or “mask” over an image of the patient and/or patient environment. The respiratory region and the regional indicator may be determined as described in U.S. patent application Ser. No. 16/713,265, which is incorporated herein by reference.

In step 604, a sensor detects sensor data, including one or more of data representing a plurality of distances between a position of the sensor and the respiratory region and captured image data. In implementations, the sensor may be a depth-sensing camera capable of detecting the data representing the plurality of distances and/or the captured image data.

In step 606, one or more misalignment qualities are generated based, at least in part, on the detected sensor data. In implementations, the one or more misalignment qualities may include one or more of an aggregate alignment metric and a sensed quality of a respiratory region.

In some embodiments, the alignment manager generates an aggregate alignment metric of the alignment between the image capture device(s) and the patient environment. In these embodiments, the alignment manager receives the generated sensor data (e.g., captured images of and/or sensed distances from an element of a patient environment, such as a respiratory region of a patient) and is operable to generate, based at least in part on the detected sensor data, an aggregate alignment metric. The aggregate alignment metric may include an aggregate distance or angular metric based on one or more of a mean, a medial, and a trimmed mean of detected distances between the image capture device(s) and the element of the patient environment. The aggregate alignment metric may account for multidimensional misalignments of different misalignment types, such as one or more of a rotational misalignment, a translational misalignment, an angle of inclination misalignment, and a distance misalignment. The aggregate alignment metric may further account for the magnitude and/or direction of the types of misalignment.

Implementations of aggregate alignment metrics may include alignment scores. In one implementation, the alignment score is determined based on an aggregate distance between the image capture device(s) and the determined respiratory region. For example, the alignment manager may be able to determine an alignment score as in equation 1. The aggregate distance may include one or more of an average, a median, and a trimmed mean of sensed distance data. In equation one, an absolute value is taken of a meter subtracted from the aggregate distance. If the distance is too different from one meter, the alignment score will reflect that the image capture device(s) are misaligned. In implementations, the alignment score will be further based on an aggregate angle, as presented in equation 2. The aggregate angle may be based on one or more of an average, median, or mean of angles detected between the image capture device(s) and the determined respiratory region or based on differences in the distance vectors by a position of the patient or of the sensor that indicate an overall aggregate angle (e.g., differing distances may indicate an overall angle of the sensor relative to the patient environment). Misalignment conditions may be based on one or more of the alignment scores. In implementations, the misalignment condition may be satisfied when the alignment scores exceed a threshold alignment score or fall outside of an acceptable range of alignment scores.

In embodiments, the alignment manager additionally or alternatively determines a sensed quality of the respiratory region. In these embodiments, the alignment manager receives detected sensor data (e.g., captured images of and/or sensed distances from an element of a patient environment, such as a respiratory region of a patient) and is configured to determine by a predefined relationship between sensor data and sensed qualities of a respiratory region, a sensed quality of the determined respiratory region based on the detected sensor data. The sensed quality may include one or more of a detected size or shape of the determined respiratory region, a detected fill ratio of an image representing the respiratory region (e.g., the extent to which there are gaps in a visual indicator overlaid over an image representing the respiratory region), a detected shape or size of a face mask wearable by a patient. The alignment manager may then determine whether the sensed quality satisfies a misalignment condition. For example, the sensed shape or size of the sensed portion of the determined respiratory region may differ depending upon the angle and/or distance from which the image capture devices sense the respiratory region. If the sensed shape or size of the respiratory region is consistent with predefined shape and/or size parameters of a misalignment condition, the sensed quality of sensed shape or size may satisfy the misalignment condition.

In implementations in which a sensed size of the portion of the detected respiratory region is used to determine misalignment, the sensed size of the respiratory region relative to the size of a comparison region (e.g., one or more of the ROI, the whole of the respiratory region, a superimposed targeting portion imparted by the sensor or image processor to the sensor data when captured) may indicate a distance between the image capture device(s) and the respiratory region. To determine the ratio may be determined based on a number of pixels in the respiratory region relative to a number of pixels in the rest of the sensed environment (or within a predefined range of the field of view of the image capture device(s)) or the relative size may be determined by integrating the depth or distance in the respiratory region or another indicated region (e.g., a region covering one or more of a patient's chest, head, arm, hand, other anatomical feature that can demonstrate orientation of the image capture device(s) relative to the patient) to generate a physical measure of the size of the indicated region.

In implementations in which the sensed shape is of a chest of a respiratory region of the patient, the sensed shape may be expected to be substantially elliptical and substantially symmetrical about a bisector of the elliptical shape. Dissimilarity in size and or shape (e.g., as elements of a misalignment condition) may be used to determine misalignment of the image capture device(s).

Additionally or alternatively, if a sensed fill ratio (ratio of distances to positions in the determined respiratory region detected that fail to satisfy an illuminating condition to present as light in a respiratory region or mask based on absolute distances to positions in the determined respiratory region or relative to other detected distances in the determined respiratory region that satisfy the illuminating condition) is an element of a misalignment condition, if the ratio falls within a predetermined range or satisfies a predetermined threshold of the misalignment condition, the sensed quality of fill ratio may satisfy the misalignment condition. In these implementations, the sensed fill ratio may be at least a part of a determination of the one or more alignment qualities.

In implementations, the predefined relationship between the generated sensor data and the sensed quality used to determine satisfaction of the misalignment condition can be represented in an inferential model such as a machine learning model. The inferential model may be pre-trained (e.g., before inferential determinations are made) based on labeled generated sensor data that is labeled as representing a sensed quality of a respiratory region. For example, the inferential model may be pre-trained by inputting received time-series data of captured images and/or generated distance signals at different times (e.g., a first time and a second time) that are labeled with the labeled sensed quality of a determined respiratory region. An inferential model trainer may then compare the output of the inferential model with the label associated with the input and determine a loss between the output and the associated label. The inferential model trainer may then backpropagate or otherwise distribute the loss through the inferential model (e.g., by adjusting one or more of weights, activation functions, and biases represented in one or more of nodes and edges of a neural network of a machine learning model). By repeating this process with different labeled generated sensor data, the inferential model can represent the predefined relationship between generated sensor data and sensed qualities.

The predefined relationship may be based on the demographics of the patient. In implementations, the predefined relationship is made specifically for the demographic of the patient by training the inferential model exclusively or predominantly on labeled data associated with the demographic. In another implementation, the inferential model is configured and/or pre-trained to take the demographic data as input, and the inferential relationships within the inferential model account for the demographic data internally.

In implementations, the labels and output of the inferential model can include data representing confidence in the output determination (e.g., probabilistic and/or statistical data). In implementations, a predefined relationship, including an inferential model, may similarly be applied between the detected sensor data and one or more aggregate alignment metrics to determine aggregate alignment metrics based on the detected sensor data, which may be pre-trained analogously using sensor data labeled with labeled aggregate alignment metrics. Further, implementations are considered where a single inferential relationship can determine, based on detected sensor data, one or more sensed qualities and one or more aggregate alignment metrics representing alignment between the image capture device(s) and the determined respiratory region.

Implementations are contemplated in which the misalignment condition includes multifactorial considerations. For example, the alignment manager may determine one or more aggregate alignment metrics and one or more sensed qualities of the respiratory region to determine whether the sensor data satisfies a misalignment condition indicating that the image capture device(s) are misaligned relative to the patient environment.

The system can additionally or alternatively determine other respiratory measurements such as values of a variety of parameters (e.g., respiratory patient motion, non-respiratory patient motion, tidal volume, minute volume, respiratory rate, etc.). In implementations, the signal processor can determine the respiratory measurements based on the generated sensor data. In other implementations, the signal processor can receive respiratory measurements generated by other means, including one or more of a transthoracic impedance measurement, an electrocardiogram, capnograph, spirometer, pulse oximeter, and a manual user entry. Implementations are also contemplated in which the signal processor does not process respiratory measurements other than the classification of patient motion. In implementations, the respiratory measurements may be accounted for in one or more of the determining of the aggregate alignment metric, determining the sensed quality, and the misalignment condition. For example, the misalignment condition may be based on a periodicity of a respiratory volume waveform. Irregular periodicity may be indicative of a misalignment, and regular waveform periodicity may be indicative of correct alignment. Similarly, respiratory rate measurements and/or associated confidence values for the respiratory rate measurements, if they are satisfactorily dissimilar to expected or predefined respiratory rate measurements (e.g., in satisfaction of a dissimilarity condition), may indicate that the image capture device(s) are misaligned relative to the patient environment.

In step 608, it is determined whether the one or more misalignment qualities satisfy a misalignment condition. The alignment manager, upon determination of one or more misalignment qualities (e.g., aggregate alignment metric and/or sensed quality), may then determine based on the one or more misalignment qualities whether the one or more misalignment qualities satisfy one or more misalignment conditions. In implementations, a misalignment condition may include a predefined threshold value and/or predefined range of values representing magnitudes and/or directions for the misalignment types. In implementations, the aggregate alignment metric may include a score that is based on values of the misalignment types. If the score is multifactorial (e.g., based on values of more than one alignment type or based on different methods of determining a nature or extent of the alignment), the score may include a weighted average or geometric mean of each of the different factors.

In an implementation, a misalignment condition may be at least partially based on a relative orientation of a region near an ROI. For example, the determined respiratory region can include an ROI of a chest region and one or more anatomical features, including, without limitation, a determined adjacent head, arm, leg, or hand. If the head is not directly above the torso in an appropriate dimension, it may indicate that the image capture device(s) are rotationally misaligned (misaligned azimuthally).

In other implementations, the misalignment conditions may include hard cutoff values for certain parameters. For example, even if the determination of satisfaction of one or more misalignment conditions is based on many factors, one or more factors can have threshold values or ranges in which the misalignment condition will automatically fail. For example, a misalignment condition may include a maximum aggregate distance of 1.5 meters between the image capture device(s) and the respiratory region. In this example, even if all other values indicate that the image capture device(s) are aligned, the misalignment condition will be satisfied solely based on the determination that the aggregate distance is 1.6 meters, ignoring all other factors. In this implementation, detecting that the determined respiratory region is more than 1.5 meters away indicates a misalignment regardless of the other factors considered.

In implementations in which the alignment manager makes determinations of one or more of aggregate alignment metrics, sensed qualities, satisfaction of misalignment conditions (or the misalignment conditions the alignment manager uses to determine the satisfaction), and misalignment classifications based on more than one of the aforementioned factors, the factors may be weighted and considered in a weighted sum or the factors may be used to determine a geometric mean as an overall misalignment score or a score for particular misalignment elements (e.g., misalignment types, directions, and/or magnitudes).

In implementations, step 608 may further include a classification operation (not illustrated) in which the one or more alignment qualities are classified. In these implementations, the alignment manager may include a misalignment classifier operable to classify a characteristic of the misalignment based on the satisfaction. The classified characteristic of misalignment may include one or more of that the image capture device(s) are misaligned relative to the determined respiratory region, one or more types of misalignment (e.g., rotational, translational, incline angle, and/or distance), and one or more magnitudes (e.g., of one or more types) of misalignment. Implementations are contemplated in which the classification is performed by a same inferential relationship as or a different inferential relationship from the inferential relationship that determines one or more of the aggregate alignment metric and/or the sensed quality of the determined respiratory region. In implementations that use the same inferential relationship, the operations of determining the sensed quality and/or aggregate metric and the operation of classification of a characteristic of misalignment may be united into a single operation (e.g., the input is sensor data, and the output is a classification of misalignment).

In step 610, an instruction is generated to provide a misalignment notification based at least in part on the satisfaction of the misalignment condition and/or a classification of the misalignment (e.g., determined based on the satisfaction of the misalignment condition). The alignment manager and/or an instruction generator of the alignment manager executable by a hardware processor of the system can generate instructions for the display of data generated by the alignment manager. The instructions can include data representing an instruction to display a misalignment notification. The misalignment notification may include one or more of an indication that the image capture device(s) and the determined respiratory region are misaligned, a type of misalignment, a magnitude of misalignment, and one or more corrective actions to take in order to align the image capture device(s) relative to the determined respiratory region.

In implementations, the instruction and/or indication may include data representing one or more of a motion classification flag, a classification-specific display, an overlaid display (e.g., configured to overlay a displayed element in a user interface to indicate a misalignment relative to the determined respiratory region), an image representation of patient motion (e.g., a visual or video representation of captured images and/or generated distance signals), an audio signal, a flashing element (e.g., where the magnitude of light of elements of a display is alternatively increased and decreased), a different alert, a code representing the aforementioned items, and the like. In implementations in which the classification is one of a number of classifications, each classification may correspond to a different display. The signal processor may output data representing the motion classification or a specific display associated in memory of the system with the alignment classification. For example, different classifications can be represented in a display by different colors, different magnitudes of light in the display, different frequencies at which to flash light in the display, and the like.

In implementations in which the misalignment classifications are representative of different degrees of misalignment or confidence in measurements thereof, the corresponding display may represent a spectrum or range of color, light magnitude, or flash frequency based on a magnitude of the one or more of the different degrees of the types of misalignment and/or different degrees of confidence in the determination of the misalignments. In one implementation, the instruction includes activating one or more lights on the image capture device(s) (e.g., an implementation is illustrated as alignment indicator 124 of the camera 114 in FIG. 1). The light display may be simple and indicate that there is a misalignment, whether by emitting different colors of light, only activating the light when the image capture device(s) are one of aligned and misaligned, only flashing a light when the image capture device(s) are one of aligned or misaligned, and the like. The display may be more complex in that it indicates one or more of a type, direction, and magnitude of misalignment and/or one or more of a type, direction, and magnitude of a corrective action to correct the misalignment. For examples of different indicator lights, see the alignment indicator system 500 of FIG. 5 and its associated description. In implementations, the magnitude of any particular type of misalignment can be represented by a frequency at which, a color of, and/or a brightness of intensity of the lights or pixels that represent a particular type or direction of misalignment or represent a particular type or direction of a corrective action to correct the misalignment.

In implementations, the instruction to provide a misalignment notification may be configured to cause a display to display the misalignment notification as an element overlaid over or underlaid under (e.g., displayed behind) another displayed respiratory measurement or an image of the patient, including an ROI image to indicate the alignment of the image capture device(s). For example, the display may be configured to display a measured respiratory rate and may display the indication of misalignment classification over or under the displayed respiratory rate. An overlaid or underlaid display of the misalignment notification may be configured to be visually contrasted from the displayed respiratory measurement. For example, the elements may be of different colors and/or may be of different transparencies, and/or the displayed misalignment notification may be at least partially transparent to maintain visibility of the displayed respiratory measurement (e.g., appearing as a highlighting of or patch over the displayed respiratory measurement). Additionally or alternatively, the displayed misalignment notification may be overlaid over an ROI image. In an alternative implementation, the instruction instructs a display not to display the respiratory measurements and/or patient images when the alignment manager determines that the image capture device(s) are misaligned.

In implementations, the misalignment manager may be communicatively coupled with a patient presence detector (e.g., hardware or software executable by one or more hardware processors) that detects whether a patient is present. In implementations in which the presence detector determines that no patient is present, the misalignment manager may one or more of deactivate itself (e.g., until a patient's presence is detected), deactivate any display or indication the misalignment manager would instruct to display an indication of misalignment, and transmit an instruction to display a different indication of alignment (e.g., color, light frequency, and/or intensity) reflecting that there is no patient present.

The above-detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations, such as the methods and processes of the various embodiments described here. The set of instructions can be in the form of a software program or application. The computer storage media can include volatile and non-volatile media, and removable and non-removable media, for storage of information such as computer-readable instructions, data structures, program modules, or other data. The computer storage media can include but are not limited to RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent that any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Additionally, the terms “comprising,” “including,” “having,” and “with” are used throughout to mean including at least the recited feature(s) such that any greater number of the same feature and/or additional types of other features are not precluded. Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may, in some cases, depend on the specific context. However, generally speaking, the nearness of completion will be so as to have the same overall result as if absolute and total completion were obtained. The use of “substantially” is equally applicable when used in a negative connotation to refer to the complete or near-complete lack of an action, characteristic, property, state, structure, item, or result. As used herein, terms such as “substantially,” “about,” “approximately,” or other terms of relative degree are interpreted as a person skilled in the art would interpret the terms and/or amount to a magnitude of variability of one or more of 1%, 2%, 3%, 4%, 5%, 6%, 7%, 8%, 9%, 10%, 11%, 12%, 13%, 14%, or 15% of a metric relative to the quantitative or qualitative feature described. For example, a term of relative degree of “about a first time” suggests the timing may have a magnitude of variability relative to the first time. When values are presented herein for particular features and/or a magnitude of variability, ranges above, ranges below, and ranges between the values are contemplated.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general-purpose microprocessors, application-specific integrated circuits (ASICs), field programmable logic arrays (FPGAs), or other equivalent integrated or discrete logic circuitry. Accordingly, the term “processor,” as used herein, may refer to any of the foregoing structures or any other physical structure suitable for implementation of the described techniques. Also, the techniques could be fully implemented in one or more circuits or logic elements.

I/we claim:

1. A method for qualifying sensor alignment relative to a patient environment, comprising:
determining a respiratory region of a patient;
detecting, by a sensor, sensor data including a plurality of distances between a position of the sensor and the determined respiratory region;
generating, based at least in part on the detected sensor data, an aggregate alignment metric;
determining that the aggregate alignment metric satisfies a misalignment condition; and
generating an instruction to provide a misalignment notification based at least partially on the determined satisfaction of the misalignment condition.

2. The method of claim 1, wherein the aggregate alignment metric includes an aggregate distance determined based on one or more of a mean, a median, and a trimmed mean of the plurality of distances.

3. The method of claim 1, wherein the aggregate alignment metric includes a first alignment score based on one or more of an aggregate distance and an aggregate angle determined based on the plurality of distances.

4. The method of claim 3, wherein the aggregate alignment metric includes an aggregate alignment score that includes a weighted average of at least the first alignment score and a second alignment score based on image data of the determined respiratory region captured by the sensor or a different sensor.

5. The method of claim 4, wherein the second alignment score is based on a predefined relationship between the image data and an alignment of the sensor or the different sensor relative to the determined respiratory region.

6. The method of claim 1, further comprising:
classifying a characteristic of misalignment of the sensor relative to the determined respiratory region at least partially based on the satisfaction of the misalignment condition, wherein the generating the instruction to provide the misalignment notification is based at least partially on the classified characteristic of misalignment.

7. The method of claim 6, wherein the characterization of misalignment includes one or more of an indication of a misalignment dimension and an indication of a magnitude of misalignment.

8. The method of claim 6, wherein the instruction includes a corrective action to modify an alignment of the sensor relative to the at least a portion of the respiratory region of the patient based on the classified characteristic of misalignment.

9. The method of claim 1, wherein the instruction is configured to cause an externally visible indicator on a sensor to provide the misalignment notification.

10. The method of claim 1, wherein the instruction is configured to cause an indicator on an external display to provide the misalignment notification.

11. A method for qualifying sensor alignment relative to a patient environment, comprising:
determining a respiratory region of a patient;
detecting, by a sensor, sensor data including one or more of a captured image of the respiratory region and a plurality of distances between a position of the sensor and the respiratory region;
determining, by a predefined relationship between sensor data and sensed qualities of the respiratory region, a sensed quality of the determined respiratory region based on the detected sensor data;
determining whether the sensed quality of the determined respiratory region satisfies a misalignment condition; and
generating an instruction to provide a misalignment notification based at least partially on the satisfaction of the misalignment condition.

12. The method of claim 11, wherein the respiratory region includes one or more of a region covering a portion of the patient's chest and a region covering at least a portion of the patient's face.

13. The method of claim 11, further comprising:
classifying a characteristic of misalignment of the sensor relative to the determined respiratory region at least partially based on satisfaction of the misalignment condition, wherein the generating the instruction to provide the misalignment notification is based at least partially on the classified characteristic of misalignment.

14. The method of claim 11, wherein the sensed quality of the determined respiratory region includes a detected measure of respiratory performance.

15. The method of claim 11, wherein the sensed quality of the determined respiratory region includes one of a detected size and a detected shape of the determined respiratory region.

16. The method of claim 11, wherein the sensed quality of the determined respiratory region includes a detected fill ratio of an image representing a mask wearable by the patient.
